(19) United States
(12) Patent Application Publication
(10) Pub. No.: US 2021/0298296 A1
(43) Pub. Date: Sep. 30, 2021

(54) METHOD OF PREVENTING MICROBES IN DISPOSABLE PRODUCTS

(71) Applicant: LOU FAYE TRADING, LLC, DORAL, FL (US)

(72) Inventor: HESHAM FAYED, DORAL, FL (US)

(73) Assignee: LOU FAYE TRADING, LLC, DORAL, FL (US)

(21) Appl. No.: 17/214,788

(22) Filed: Mar. 26, 2021

Related U.S. Application Data
(60) Provisional application No. 63/001,231, filed on Mar. 27, 2020.

(51) Int. Cl. A01N 31/06; A61F 13/20; A61F 13/15; A61L 15/46; A61L 15/40; A61L 15/28; A61L 2/18; A61L 2/22; A61L 9/014; A41D 13/11; A47G 9/02; A47G 11/00

(52) U.S. Cl. CPC A01N 31/06; A61L 2101/34; A61F 13/15731; A61L 15/46; A61L 15/40; A61L 15/28; A61L 2/18; A61L 2/22; A61L 9/014; A41D 13/1192; A47G 9/0238; A47G 11/003; A61L 2300/216; A61L 2420/02; A61F 13/2085

(57) ABSTRACT

The present invention describes methods and product for protection against microbial colonization associated with the use of hygienic products, such as diapers, bandages, sanitary napkins, tampons, gowns, facemasks, table covers, and the like, through significantly minimizing, inhibiting, or preventing microbial colonization in the hygienic product material. The present invention provides for using a hygienic product containing at least one inner layer of its composition that is integrated with an effective amount of natural, viable, non-pathogenic, antimicrobial, Terpinen-4-ol, for inhibiting the growth and multiplication of microorganisms. The method used for integrating the natural antimicrobial product, the Terpinen-4-ol, to the selected sanitary product layer utilizes, amongst others, a nanotechnology process.

### FIELD OF INVENTION

The present invention discloses a method to provide antibacterial properties to standard disposable products commonly used for human hygiene, such as, for example and without limitation, sanitary products, clothing articles, and other disposable and reusable products that are used to filter pathogens from the air, products placed in a health provider facility environment, and products that come in contact with a human.

### BRIEF DESCRIPTION

Disposable sanitary products and garments used by humans, especially if not frequently changed, trap temperature and moisture that create ideal conditions to stimulate the colonization of the bacterial flora transmitted from the user's skin threatening the trigger of an infection in the genital area and urinary tract. Also during menstruation the alteration of the microbial flora contributes as an added factor. Further physical contact and aerobiology transmission may trap pathogens in disposable materials used in health-provider facilities and gathering locations such as, for example and without limitation, schools, restaurants, nurseries, cruises, airplanes, buses, and the like.

### SUMMARY OF THE INVENTION

The present invention provides a method for protection against microbial colonization in disposable products, utilized for human hygiene, including air filtering purposes, consequently protecting its users from physical and aerobiology contagion. The invention involves integrating a natural substance with proven antimicrobial efficacy, Terpinen-4-ol or similar, in a water soluble or petroleum derivate solution that is diluted according to the intended purpose and strength desired corresponding to the final application of the product, as it is well known to a person skilled in the art, and applied to the fibers of at least the first fast absorbent layer of the product using a variety of methods, including but not limited to nanotechnology implanting.

The antimicrobial efficacy of Terpinen-4-ol is tested in many published in vivo and in vitro clinical trials against many organisms including gram negative, gram positive, fungus, viral and protozoa organisms thereof protects against possible common genital and urinary tract infections. Utilizing nanotechnology process to bind and integrate the natural antimicrobial product to the fibers of the selected layer of the product at a molecular level rendering the coating firmer with longer lasting effect with no interference with the absorption function of the absorbent layer.

### BACKGROUND OF THE INVENTION

In 2006 a patent was published (20060177429 dated Aug. 10, 2006) describing a claim of integrating living harmless bacteria (lactic acid producing probiotics) of non-pathogenic Bacillus coagulans targeting the user's skin during the use of a sanitary product, thereby inhibiting or preventing skin infection caused by Staphylococcus species or a Streptococcus species.

The invention targeted direct transmission and delivery of the lactic acid producing living harmless bacteria to the user skin.

There were neither indications provided on the minimum maintained Colony Forming Units (cfu) of the probiotic used nor any indications of the viability of the bacteria over any specific time.

Our invention targets the sanitary product itself to render its components hostile to common gram negative, gram positive, fungi, viruses, and parasites providing protection to the user especially if the product is not frequently changed. The antimicrobial material used, Terpinen-4-ol, is a 100% a natural product of proven lethal efficacy to wide range of gram positive, gram negative, fungus, and protozoa organisms.

The Terpinen-4-ol is integrated with the fibers of at least the first absorbent layer at the molecular level using a nanotechnology process thus ensuring maximum performance for longer time. Integration with other layers of the hygienic product may provide additional protection, in particular where the folds of the product can create an ideal environment for bacteria reproduction.

In 2007 a patent was published (EP1622652 B1), the publication pertains to hygiene products, such as sanitary napkins, diapers, panty liners, tampons, incontinence guards, hygiene tissues and the like, which comprise a probiotic composition comprising a bacterial preparation of at least one lactic acid producing bacterial strain and a contact sorption drying carrier dispersed in a lipid phase. The disclosed invention also pertains to a method for producing a hygiene product comprising lactic acid producing bacteria, dried with the aid of contact sorption drying carriers, in a lipid phase. The invention provides a manufacturing process that has advantages as regards to economy, simplicity, and bacterial survival during manufacturing and subsequent storage

The invention target direct transmission and delivery of the lactic acid producing living harmless bacteria to the user's skin.

There were neither indications provided on the minimum maintained Colony Forming Units (cfu) of the probiotic used nor any indications of the viability of the bacteria over any specific time.

Our invention targets the sanitary product itself to render its components hostile to common gram negative, gram positive, fungi, viruses, and parasites providing protection to the user especially if the product is not frequently changed. The antimicrobial material used, Terpinen-4-ol, is a 100% a natural product of proven lethal efficacy to wide range of gram positive, gram negative, fungus and protozoa organisms.

The Terpinen-4-ol is integrated with the fibers of at least the first absorbent layer at the molecular level preferably using a nanotechnology process thus ensuring maximum performance for longer time.

None of the above disclosed invention utilizes the nanotechnology our invention uses to ensure efficacy on longer time.

### DRAWINGS

FIG. 1 depicts a plurality of hygienic products used humans, shown as examples and without limitations, that may come in contact with human skin and with pathogens, such as sanitary napkins, dippers, shoe covers, face masks, lab coats, bed sheets, curtains, tablecloths, gowns, lab coats, etc.

FIG. 2 depicts a sanitary pad showing the different constructions layers, having the top layer, which makes contact with human skin, coated/impregnated/integrated with a solution of Terpinen-4-ol.

### DETAILED DESCRIPTION OF THE INVENTION

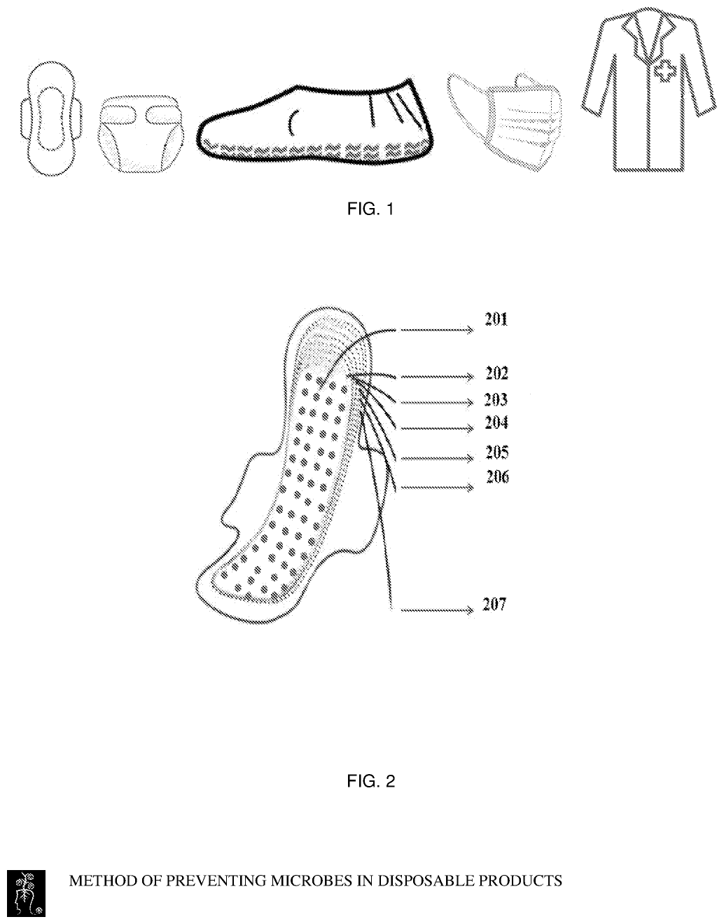

The present invention provides antimicrobial protection to hygienic sanitary products, such as those depicted in FIG. 1 and FIG. 2, through inhibiting or preventing the transmission of bacterial flora and other microorganism from the user to the product.

FIG. 2 depicts the layers of a sample product, such as a sanitary pad, that includes an innermost layer (201) in which Terpinen-4-ol is integrated into its fibers or coated on the surface of the innermost layer. Other layers of the sanitary pad, such as air laid paper wrapped (202), side leak layer wrap (203), polymer layer (204), air laid paper wrap (205), comfortable bottom layer (206) and back adhesive layer (207) may also be coated, soaked, sprays, of nanotechnology imbued with a solution of Terpinen-4-ol for additional protection.

The method used to inhibit or prevent pathogen colonization consists of integrating a natural liquid product, a Terpinen-4-ol solution, with tested antimicrobial properties, to the first absorbent layer of the product, as shown in FIG. 1, at a molecular level using a nanotechnology process or other implantations processes well known to a person skilled in the art.

The coating material used is a water or petroleum derivate solution with a diluted concentrate of natural Terpinen-4-ol, derived from natural Tea Tree Oil, incorporated at least into the first absorbent layer of the sanitary product during the manufacturing process using selected nanotechnology process, such as, for example and without limitation, high pressure cold spray or similar.

The concentration/dilution level of Terpinen-4-ol varies according various factors including the substrate's material absorption and retention qualities, use and purpose of the treated product, climate (temperature, humidity, light exposure, etc.), working conditions where the product will be used, time of product serviceability, and process of integration. The solution's concentration level of Terpinen-4-ol may vary from a 0.25% to 95%.

The combined product, coated on the fibers of the first absorbent layer at molecular level, inhibits the transmission of skin flora, or any other organism, of the user in contact with the first layer, from colonizing through its mechanism of action of damaging and losing the organism's cytoplasmic contents leading to the destruction of the microorganism, therefore eliminating a possible source of infection.

### Chemical Composition of Terpinen-4-ol Precursor

Terpinen-4-ol is derived from Tea Tree Oil, also known as Melaleuca Oil; it is steam-distilled primarily from the leaves of M. alternifolia. The leaves contain 2% of a pale-yellow volatile oil. Approximately one-third of the essential oil fraction is composed of terpene hydrocarbons, such as beta-pinene, p-cymene, limonene, aromadendrene, 1-8 cineole, and many others. The remaining portion of the essential oil fraction is composed of oxygenated terpenes, with 30% made up of Terpinen-4-ol.

Terpinen-4-ol appears responsible for most of the antimicrobial activity of Tea Tree Oil. Terpinolene (1%), alpha-terpineol (1%), and alpha-terpinene, are other abundant terpenes present. The Australian standard “Oil of Melaleuca contains 30% -40% Terpinen-4-ol and less than 15% cineole. Tea Tree Oils with high cineole content are thought to be of poor quality and more likely to cause skin irritation. Our invention utilizes only Terpinen-4-ol.

### Terpinen-4-ol

Comprising greater than 30 percent Terpinen-4-ol Is the highest the percentage of Terpinen-4-ol in Tea Tree Oil and is considered the highest of the quality of the product. The Terpinen-4-ol, the major chemical constituent of Tea Tree Oil is believed to have most of the antimicrobial activity.

Researchers from Australia have found that only the Terpinen-4-ol of the water-soluble components in Tea Tree Oil will suppress the inflammatory response in cells activated by monocytes. This means that it is this chemical that has the greatest anti-inflammatory properties against the cells in the body which first respond to injury.

Terpinen-4-ol also appears to inhibit the growth of human melanoma cells in a test tube. Lead researcher Annarica Calcabrini published the team's findings in a 2004 issue of the “Journal of Investigative Dermatology.” They found that the Terpinen-4-ol was able to impair the growth of human melanoma cells.

### 1,8-Cineole

Tea Tree Oil products should contain less than 10 percent cineole, according to www.drugs.com. This is the ingredient that can cause irritation to the skin and internal CNS toxicity. This chemical is a colorless liquid that is “stable, flammable and incompatible with acids, bases and strong oxidizing agents,” according to the Material Data Safety Sheet. It is harmful if ingested.

### Pinene

Pinene is an organic [symbol] compound, is a major component in Tea Tree Oil and responsible for its pine odor, according to the University of Minnesota's Biocatalysis and Biodegradation Database. It is a member of the terpenes family of chemicals and is widely used in medicines to treat acne, as well as in deodorants and some flavorings in small amounts.

### Chemical composition of Terpinen-4-ol

Formula: C₁₀H₁₈O
Molecular Weight: 154.2493
IUPAC Standard InChI

InChI1=S/C10H180/c1-8(2)10(11)6-4-9(3)5-7-10/h4,8,11H,5-7H2,1-3H3

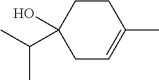

(+)-Terpinen-4-ol

### Stereoisomers: 3-Cyclohexen-1-ol, 4-methyl-1-(1-methylethyl)-, I-

Other names: 3-Cyclohexen-1-ol, 4-methyl-1-(1-methylethyl)-; p-Menth-1-en-4-ol; 1-Terpinen-4-ol; 4-Carvomenthenol; 4-Terpineol; 1-Methyl-4-isopropyl-1-cyclohexen-4-ol; 4-Terpinenol; Terpene-4-ol; Terpinene-4-ol; para-Menth-1 -en-4-ol; 1-para-Menthen-4-ol; Terpinenol-4; Terpinenolu-4; 3-Cyclohexen-1 -ol, 4-methyl-1[methylethyl]-; 4-methyl-1-(1-methylethyl)-3-cyclohexen-1-ol; 4-methyl-1-isopropyl-3-cyclohexen-1-ol; 4-Methyl-1-(methylethyl)-3-cyclohexen-1-ol; Terpin-4-ol; Terpine-4-ol; Terpineol-4; (.+/−.)-p-Menth-1-en-4-ol; NSC 147749; 1-methyl-4-isopropyl-1-cyclohexen-4-ol (4-terpineol); α-Terpinen-4-ol; Terpin-4-en-1-ol; Terpinen-4-ol [4S-(+), 4R-(−)]; 1-p-Menthen-4-ol; 4-Terpeneol; I-4-terpineol

### Medicinal Uses

### Antifungal Effects

Recent evidence supports the use of Tea Tree Oil through its Terpinen-4-ol contents to treat microbial infection of the skin and mucosa. Since 1992, several well-controlled clinical trials, some directly comparing Tea Tree Oil with a conventional treatment, have been published. A randomized, double-blind trial in 104 patients with tinea pedis compared 10% w/w Tea Tree Oil cream with 1% tolnaftate and placebo. Tolnaftate is a thiocarbamate synthetic antifungal that has an 80% cure rate for tinea pedis. Both treatment groups showed significant improvements in clinical symptoms; however, only tolnaftate showed conversion to negative culture at the end of therapy.

In a double-blind, multicenter, randomized, controlled trial involving 117 patients with nail fungus (onychomycosis), infections were treated for six months with twice-daily topical applications of either 1% clotrimazole or 100% Tea Tree Oil. At the end of treatment, both groups showed similar positive results. There was a decrease in fungus in cultures as well as a clinically documented resolution of the infection with both products. Tea Tree Oil has also been effective in eliminating head lice (Pediculus humanus capitis) when applied in an alcoholic solution to the scalp. Growth of Pityrosporum ovale, the organism that causes seborrheic dermatitis and dandruff, appears to be inhibited by Tea Tree Oil. Topical scalp preparations with the oil may be effective in this condition, but clinical trials have not been conducted.

Several in vitro studies have revealed that Tea Tree Oil inhibits the growth of many species and strains of fungi and yeast. The antifungal activity of Tea Tree Oil (0.5%) was tested against 26 strains of dermatophytes and 54 yeast strains (including 32 strains of Candida albicans and 22 strains of Malassezia furfur). Tea Tree Oil inhibited the growth of all of these fungal strains. In another study, the susceptibility of 64 M. furfur strains to Tea Tree Oil was examined. For 90% of the strains, the minimum inhibitory concentration of Tea Tree Oil was 0.25%. In another study, Tea Tree Oil inhibited the growth of C. albicans, Trichophyton rubrum, Trichophyton mentagrophytes, Trichophyton tonsurans, Aspergillus niger, and Microsporum gypsum. Two reports indicate successful treatment of vaginal yeast infections with Tea Tree Oil. Treatment required four weeks for yeast eradication and relief of symptoms.

In vivo activity of Terpinen-4-ol, the main bioactive component of Melaleuca alternifolia Cheel (Tea Tree) oil against azole-susceptible and -resistant human pathogenic Candida species.

### Antibacterial Effects

In vitro studies show the effectiveness of Tea Tree Oil in inhibiting several common skin pathogens. Terpinen-4-ol and whole Tea Tree Oil were found to be equally effective for activity against Staphylococcus aureus. Several major components of Tea Tree Oil (Terpinen-4-ol, alpha-terpineol, alpha-pinene, and cineole) tested for their effects against S. aureus, Staphylococcus epidermidis, and Propionibacterium acnes. Except for cineole, all of the constituents tested were inhibitory to all three organisms.

P. acnes is the major bacterium that causes acne vulgaris. The effectiveness and skin tolerance of Tea Tree Oil as a treatment was evaluated in a single-blind, randomized clinical trial involving 124 patients. Treatment efficacy of a 5% Tea Tree Oil gel was compared with that of a 5% benzoyl peroxide lotion. Both treatments significantly reduced the number of inflamed and non-inflamed lesions. Tea Tree Oil had a much slower onset of action but also had fewer adverse side effects, such as skin scaling, dryness, and irritation.

In an in vitro study, the susceptibility of 66 isolates of S. aureus to Tea Tree Oil was investigated. All of the isolates were inhibited by Tea Tree Oil, with a minimum concentration of 0.25%. In another study, Burnaid, a sorbalene-based cream containing 40 mg/g of Tea Tree Oil as well as 1 mg/g of triclosan, was tested for inhibitory effects on several infectious microorganisms common in burn patients: 15 Enterococcus faecalis, S. aureus, Escherichia coli, and Pseudomonas aeruginosa. Burnaid significantly inhibited only S. aureus and E. coli.

Another recent study demonstrated that Tea Tree Oil stimulated autolysis in stationary phase cells of E. coli. Electron micrographs of cells grown in the presence of Tea Tree Oil showed the loss of electron-dense material, formation of extracellular blebs, and coagulation of cell cytoplasm.

The December 2009 issue of “Letters in Applied Microbiology” published a study investigating the antiviral activity of TTO and its main component, Terpinen-4-ol. These compounds were evaluated for their inactivating effects against several viruses; including, polio type 1, ECHO 9, Coxsackie B1, adeno type 2, and herpes simplex (HSV) type 1 and 2. The results of the study demonstrated that Tea Tree Oil and some of its constituents possess inhibitory effects on influenza virus subtype H1N1. However, all the compounds tested were ineffective against polio 1, adeno 2, ECHO 9, Coxsackie B1, HSV-1 and HSV-2. It was concluded that Tea Tree Oil has an antiviral activity against influenza virus subtype H1N1 only, principally attributed to Terpinen-4-ol, and Tea Tree Oil is a promising drug in the management of influenza infections.

A follow-up study was published in the January 2011's issue of “Antiviral Research.” Here, the study investigated the action of Tea Tree Oil and its active components against different steps of the replicative cycle of influenza virus subtype H1N1 in dog kidney cells at different times after infection. These experiments showed that viral replication was significantly inhibited if Tea Tree Oil was added within two hours after infection of the cells, which indicated interference at the beginning of the viral replicative cycle during the adsorption step, or the actual entering of the virus into the host cell. The results suggest that Tea Tree Oil did not interfere with attachment of the virus to the cell.

The November 2008 issue of “Complementary Therapies in Clinical Practice” detailed the first clinical study in which Tea Tree Oil was used for the successful treatment with of a pediatric patient with warts on her right middle finger. The clinicians applied Tea Tree Oil topically to the infection once daily for 12 days and found complete viral clearance of the infected areas. This study emphasizes the potential use of Tea Tree Oil in the treatment of common warts due to human papilloma virus.

An article appearing in the January 2004 issue of “Phytotherapy Research” contained a study of essential oils from fresh leaves of several related species of the genus Melaleuca. The oils were distilled, analyzed and rated on efficacy as antimicrobials and antivirals against Herpes simplex virus type 1, HSV-1, the causative agent of oral and genital herpes in humans. The antiviral properties of these oils were studied in African green monkey kidney cells infected with HSV-1 and found to be an effective treatment by inhibiting the replication of viral particles and preventing infection of surrounding cells.

The following constituents were the most commonly found compounds in essential oils with antiprotozoal activity: Thymol, which appears in L. alba, L. ciriodora, L. dulcis, L. micromera, L. origanoides, O. basilicum, and T. vulgaris; eugenol, which was found in O. basilicum, O. gratissimum. O. sanctum, and S. aromaticum; camphor, which was present in A. absinthium and T. hirtus; carvacrol, which was a constituent of L. alba, O. virens, and T. capitata; and Terpinen-4-ol, which was a component of the Tea Tree Oil (M. alternifolia), M. officinalis and T. vulgaris.

The antiviral activity of Tea Tree Oil was first shown using tobacco mosaic virus and tobacco plants. In field trials with Nicotiniana glutinosa, plants were sprayed with 100, 250, or 500 ppm Tea Tree Oil or control solutions and were then experimentally infected with tobacco mosaic virus. After 10 days, there were significantly fewer lesions per square centimeter of leaf in plants treated with Tea Tree Oil than in controls. Next, Schnitzler et al. examined the activity of Tee Tree Oil and eucalyptus oil against herpes simplex virus (HSV). The effects of Tea Tree Oil were investigated by incubating viruses with various concentrations of Tea Tree Oil and then using these treated viruses to infect cell monolayers. After 4 days, the numbers of plaques formed by Tea Tree Oil-treated virus and untreated control virus were determined and compared. The concentration of Tea Tree Oil inhibiting 50% of plaque formation was 0.0009% for HSV type 1 (HSV-1) and 0.0008% for HSV-2, relative to controls. These studies also showed that at the higher concentration of 0.003%, Tea Tree Oil reduced HSV-1 titers by 98.2% and HSV-2 titers by 93.0%. In addition, by applying Tea Tree Oil at different stages in the virus replicative cycle, Tea Tree Oil was shown to have the greatest effect on free virus (prior to infection of cells), although when Tea Tree Oil was applied during the adsorption period, a slight reduction in plaque formation was also seen. Another study evaluated the activities of 12 essential oils, including Tea Tree Oil, for activity against HSV-1 in Vero cells. Again, Tea Tree Oil was found to exert most of its antiviral activity on free virus, with 1% oil inhibiting plaque formation completely and 0.1% Tea Tree Oil reducing plaque formation by approximately 10%. Pretreatment of the Vero cells prior to virus addition or post treatment with 0.1% Tea Tree Oil after viral absorption did not significantly alter plaque formation.

Some activity against bacteriophages has also been reported, with exposure to 50% Tea Tree Oil at 4° C. for 24 h reducing the number of SA and T7 plaques formed on lawns of S. aureus and E. coli, respectively.

The results of these studies indicate that Tea Tree Oil may act against enveloped and non-enveloped viruses, although the range of viruses tested to date is very limited.

Two publications show that Tea Tree Oil has antiprotozoal activity. Tea Tree Oil caused a 50% reduction in growth (compared to controls) of the protozoa Leishmania major and Trypanosoma brucei at concentrations of 403 mg/ml and 0.5 mg/ml, respectively. Further investigation showed that Terpinen-4-ol contributed significantly to this activity. In another study, Tea Tree Oil at 300 mg/ml killed all cells of Trichomonas vaginalis. There is also anecdotal in vivo evidence that Tea Tree Oil may be effective in treating Trichomonas vaginalis infections.

### Nanotechnology Integration

In recent years nanotechnology has become one of the most important and exciting forefront fields in physics, chemistry, engineering, and biology. Nanotechnology deals with various structures of matter having the dimension of the order of a billionth of a meter. Structures on this scale have been shown to have unique and novel functional properties.

Based on that principle, many applications of nanotechnology from the simple to the complex have been done. One of these applications is to prepare antimicrobial textiles and nonwoven material in their nanoscale.

Particles at the nanoscale are below the wave length of visible light and therefore, cannot be seen. Consequently they can impart new properties. For example, Ti-nanoparticles are applied for the textile materials in order to develop textile products with UV-protection and self cleaning property. Also Silver nanoparticles are used as antimicrobial agent for wound-dressing materials as well as for wound healing. In addition, the production of fibers with diameter less than 100 nm is now feasible with the invention of electrospinning process.

Electrospinning is a manufacturing new technology capable of producing thin, solid, polymer strands from solution by applying a strong electric field to a spinneret with a small capillary orifice. The spun, polymer based, nanofibers, can be loaded with different additives.

The resulted nanofibers are collected and bundled. These electrospun fibers have high surface area and porous structure, where more than one drug can be encapsulated directly into the fiber. The resulted matrix can be used extensively for sanitary products production with multifunctional properties. Currently there are many technologies available to process the nano-binding with proven efficacy.

### Dosage Used

The used of Terpinen-4-ol solution in concentration ranging from 0.25% - 95%, depends on several factors, including but not limited to, the substrate's material absorption and retention properties, the use and purpose of the treated product, the climate (temperature, humidity, light exposure, etc.), working conditions where the product will be used, time of product serviceability, and process of integration.

The solution's concentration level of Terpinen-4-ol may vary from a 0.25% to 95% matches the concentration demonstrated in vitro and in vitro antimicrobial activity.

### Methods

The Terpinen-4-ol concentrate of 0.25% - 95% using standard dilution methods is integrated at the molecular level with the fiber material of the first absorbent layer of the product.

The integration utilizes any available nanotechnology process, such as high pressure cold spray or similar known to those skilled in the art; the molecular integration ensures long term performance with no effect on the absorption capability of the layer.

### Processing

The manufacturing process follows the standard methods used to manufacture products that are intended to be treated with the Terpinen-4-ol solution. The first layer integration process can be processed at the same manufacturing site or processed at designated service provider and shipped to the product manufacturing site.

What is claimed is:

1. A method for preventing or inhibiting microbes from colonizing hygienic products that come in contact with human skin, the method comprising Integrating a solution of Terpinen-4-ol into at least one layer of the hygienic product.

2. The method of claim 1, wherein the at least one layer of a hygienic product includes the innermost layer that comes in contact with a human's skin.

3. The method of claim 1, wherein the at least one layer is a layer made of an absorbent material selected from a group consisting of cotton, linen, wool, silk, paper, cellulose, manmade fibers, and a combination thereof.

4. The method of claim 1, wherein the hygienic product is at least one select from a group consisting of sanitary napkins, tampons, diapers, sanitary pads, towels, bed sheets, socks, gowns, head covers, face masks, bed protectors, tablecloths, underwear, handkerchiefs, wound dressings, protective gauze, articles of clothing, and wash cloths.

5. The method of claim 1, wherein the solution of Terpinen-4-ol consist of a dilution of Terpinen-4-ol in a concentration range of 0.25% to 95%.

6. The method of claim 1, wherein integrating a solution of Terpinan-4-ol into the at least one layer of a hygienic product is accomplished through at least one method selected from a group consisting of soaking, spraying, coating, nanotechnology integration, electrospinning, and weaving.

7. The manufacture of a microbe inhibited or resistant product comprising the steps manufacturing the product and placing at least one layer of a material that has been integrated with a solution of Terpinen-4-ol as the innermost layer that comes into contact with a human's skin or used as an air filter.

8. The method of claim 7, wherein the product is at least one select from a group consisting of sanitary napkins, tampons, diapers, sanitary pads, towels, bed sheets, socks, gowns, head covers, bed protectors, tablecloths, wash cloths, underwear, handkerchiefs, wound dressings, protective gauze, articles of clothing, stand alone and device integrated air filters, face masks, and surgical masks.

9. The method of claim 7, wherein the material is an absorbent material selected from a group consisting of cotton, linen, wool, silk, paper, cellulose, manmade fibers, and a combination thereof.

10. The method of claim 7, wherein the solution of Terpinen-4-ol consist of a dilution of Terpinen-4-ol in a concentration range of 0.25% to 95%.

11. The method of claim 7, wherein integrating a solution of Terpinan-4-ol into the innermost layer is accomplished through at least one method selected from a group consisting of soaking, spraying, coating, nanotechnology integration, electrospinning, and weaving.

12. A microbe inhibited or resistant product, the product consisting of standard materials, a plurality of construction structures, and has at least one layer, the innermost layer that comes in contact with human skin or filters air, which integrates a solution of Terpinen-4-ol.

13. The microbe inhibited or resistant product of claim 12, wherein the product is at least one select from a group consisting of sanitary napkins, tampons, diapers, sanitary pads, towels, bed sheets, socks, gowns, head covers, bed protectors, tablecloths, wash cloths, underwear, handkerchiefs, wound dressings, protective gauze, articles of clothing, stand alone and device integrated air filters, face masks, and surgical masks.

14. The microbe inhibited or resistant product of claim 12, wherein the standard material is one selected from a group consisting of cotton, linen, wool, silk, paper, cellulose, manmade fibers, and a combination thereof.

15. The microbe inhibited or resistant product of claim 12, wherein the solution of Terpinen-4-ol consist of a dilution of Terpinen-4-ol in a concentration range of 0.25% to 95%.

16. The microbe inhibited or resistant product of claim 12, wherein integration a solution of Terpinan-4-ol into the at least one layer of the product is accomplished through at least one method selected from a group consisting of soaking, spraying, coating, nanotechnology integration, electrospinning, and weaving.
